(19) United States
(12) Patent Application Publication
(10) Pub. No.: US 2025/0302863 A1
(43) Pub. Date: Oct. 2, 2025

(54) USE OF THBS1 INHIBITOR FOR OVERCOMING DRUG RESISTANCE IN CANCER

(71) Applicant: KOREA ADVANCED INSTITUTE OF SCIENCE AND TECHNOLOGY, Daejeon (KR)

(72) Inventors: Kwang Hyun CHO, Daejeon (KR); SooMin LEE, Daejeon (KR); Hyun Young HAN, Daejeon (KR)

(21) Appl. No.: 18/840,605

(22) PCT Filed: Mar. 23, 2023
(86) PCT No.: PCT/KR2023/003905

§ 371 Date: Aug. 22, 2024

(30) Foreign Application Priority Data
Mar. 23, 2022 (KR) 10-2022-0036220

(51) Int. Cl. A61K 31/7105; A61K 31/17; A61K 31/404; A61K 31/439; A61K 38/00; A61P 35/00; C07K 16/18; G01N 33/50

(52) U.S. Cl. CPC A61K 31/7105; A61K 31/17; A61K 31/404; A61K 31/439; A61K 38/00; A61P 35/00; C07K 16/18; G01N 33/5011; G01N 2500/10

(57) ABSTRACT

The present invention relates to a use of THBS1 as a novel combination drug target that can overcome drug resistance of a targeted anticancer agent. A THBS1 inhibitor according to the present invention inhibits the drug resistance of a target anticancer agent and thus increases an anticancer effect when administered in combination with a target anticancer agent. Accordingly, the present invention can overcome resistance to a targeted anticancer agent and increase a treatment success rate of anticancer drugs for cancer patients, thereby suggesting new possibilities for treatment strategies using targeted anticancer drugs and contributing to the realization of precision medicine.

### SEQUENCE LISTING

This application includes a Sequence Listing in the XML file in .XML format that is electronically submitted via EFS-Web on Apr. 16, 2025. The XML file contains a sequence listing entitled “1009082107US9SequenceListing.xml” created on Apr. 16, 2025, and is 7,255 bytes in size. The Sequence Listing contained in this 1009082107US9SequenceListing.xml file is part of the specification and is hereby incorporated by reference herein in its entirety.

### TECHNICAL FIELD

The present invention relates to a use of THBS1 as a novel combination drug target that can overcome drug resistance of a targeted anticancer agent.

### BACKGROUND ART

Cancer is a representative prevalent and incurable disease and is becoming a major socioeconomic problem. According to the ‘Draft of Cancer Prevention and Control Plan’ published by the International Agency for Research on Cancer (IARC) under the World Health Organization (WHO) in 2017, the number of cancer patients has increased significantly worldwide, and the annual number of cancer cases is estimated to approach 22 million by 2030, which is increased by approximately 54% compared to 2012, and in 2010 alone, the costs used for cancer diagnosis and treatment approached KRW 1,315 trillion worldwide. Even in Korea, according to ‘Cancer registration statistics of the Ministry of Health and Welfare’ published in 2014, the cancer incidence rate has steadily increased since 2000, and in 2010, the number of cancer patients exceeded 200,000 (about 400 per 100,000 people). According to the ‘Death cause statistics of Statistics Korea’ published in 2015, the highest increased cause of death in mortality over the past 10 years is cancer, and the number of cancer deaths reached 76,975 (about 150.8 per 100,000 people), approximately 27.9% of all deaths in 2015.

Recently, various targeted anticancer agents have been developed for cancer treatment. Since the targeted anticancer agent attacks a specific target expressed only in cancer cells, the targeted anticancer agent may dramatically increase therapeutic effects while reducing side effects. However, despite great expectations, the effect of the targeted anticancer agent is limited in clinical practice due to cancer resistance to drugs. In particular, due to the complicated dynamics of signaling networks that intertwin with a feedback regulatory relationship, cancer has adaptive resistance that can adapt to and offset a perturbation effect caused by drugs. This drug resistance fundamentally inhibits the efficacy of targeted therapeutic agents. As such, a network approach is needed to find the principles of drug resistance in cancer cells, in which many signaling networks are comprehensively involved, and to identify biomarkers for resistant patient groups and discover parallel treatment targets to overcome resistance.

Accordingly, many targeted anticancer agents have been developed to inhibit mutated signaling pathways. Since human cancer cells respond differently to anticancer agents depending on the presence or absence of mutations, it is important to determine which mutation the cancer cells have and use appropriate anticancer agents thereto. In particular, lose-of-function mutations of p53, a representative tumor suppressor gene, are commonly found in a significant number of cancer patients, especially lung cancer.

Currently, the targeted anticancer agent is administered alone or in combination with chemotherapy to treat cancer, but treatment is more difficult for patients who are resistant to targeted anticancer agents. Therefore, it is necessary to discover target genes that play a major role in overcoming resistance to an anticancer agent and determine resistance occurrence mechanisms related thereto.

About 50% of human cancers show inactivation of p53 protein functions through mutations in the p53 gene or defects in the mechanism of activating p53. Such a disorder to the p53 function plays a critical role in tumor evolution by allowing avoidance from p53-dependent responses. Many recent studies have focused on directly targeting p53 mutations by identifying selective low-molecular compounds to significantly reduce p53 mutations or restore the tumor suppressive function of p53.

In particular, p53 mutations are the most common in lung cancer, and when these p53 mutations exist, the prognosis is known to be worse. A targeted anticancer agent, such as PRIMA-1MET (APR-246), shows high therapeutic potential as an anticancer agent by reactivating p53 in the presence of p53 mutation, but in many cases, resistance remains even after treatment with the drug.

Accordingly, it is possible to enhance the treatment effect by presenting a target that can overcome resistance to targeted anticancer agents such as APR-246 that activates p53 as a new combination therapeutic agent for cancer patients with a p53 mutation, one of the most important mutations in cancer.

In other words, if the function of the p53 gene may be restored, it can be an effective and promising cancer treatment strategy, which will contribute to realizing personalized medicines and improving patient survival rates and quality of life due to unnecessary anticancer agent treatment through targeted treatment.

### DISCLOSURE

### Technical Problem

Under these situations, the present inventors have made extensive research efforts to discover combination drug targets that can achieve excellent therapeutic activity to cancers and overcome the therapeutic limitations of targeted anticancer agents. As a result, the present inventors identified the THBS1 gene as a combination target that can increase the drug responsiveness of cancers that are resistant to a p53-activating drug due to loss-of-function mutations that occurred in the p53 gene, a tumor suppressor gene. In addition, the present inventors identified that the THBS1 was inhibited as a combination treatment target to overcome drug resistance to a targeted anticancer agent to not only overcome the resistance to the targeted anticancer agent, but also increase the efficacy of the targeted anticancer agent, and then completed the present invention.

Therefore, an object of the present invention is to provide a composition for inhibiting resistance to an anticancer agent including a THBS1 inhibitor as an active ingredient.

Another object of the present invention is to provide a composition for enhancing responsiveness to an anticancer agent including a THBS1 inhibitor as an active ingredient.

Yet another object of the present invention is to provide an anticancer adjuvant including the composition.

Yet another object of the present invention is to provide a pharmaceutical composition for preventing or treating cancer including a THBS1 inhibitor and an anticancer agent as active ingredients.

Yet another object of the present invention is to provide a food composition for inhibiting resistance to an anticancer agent including a THBS1 inhibitor as an active ingredient.

Yet another object of the present invention is to provide a screening method of substances for inhibiting resistance to an anticancer agent.

Yet another object of the present invention is to provide an information providing method for determining resistance to a p53 activator.

Yet another object of the present invention is to provide a method for inhibiting resistance to an anticancer agent including administering to a subject a composition for inhibiting resistance to an anticancer agent including a Thrombospondin 1 (THBS1) inhibitor as an active ingredient.

Yet another object of the present invention is to provide a treatment method including administering to a subject a pharmaceutical composition for preventing or treating cancer including a THBS1 inhibitor and an anticancer agent as active ingredients.

### Technical Solution

The terms used herein are used for the purpose of description only, and should not be construed to be limited. The singular expression includes the plural expression unless the context clearly dictates otherwise. In the present invention, it should be understood that the term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which exemplary embodiments pertain. Terms which are defined in a generally used dictionary should be interpreted to have the same meaning as the meaning in the context of the related art, and are not interpreted as ideal or excessively formal meanings unless otherwise defined in the present invention.

Hereinafter, the present invention will be described in more detail.

According to an aspect of the present invention, the present invention provides a composition for inhibiting resistance to an anticancer agent including a Thrombospondin 1 (THBS1) inhibitor as an active ingredient.

The THBS1 of the present invention is a combination target gene for overcoming drug resistance, which is derived by key positive feedback to a drug resistance mechanism of an anticancer agent through simulation of a discovery model of combination targets to overcome resistance to a targeted anticancer agent (drug, chemical) according to the present invention. It was confirmed that when the expression of THBS1 was inhibited, resistance to an anticancer agent was suppressed, sensitivity to anticancer agents was increased, and the cell viability of anticancer agent-resistant cell lines was decreased. Therefore, the present invention relates to an excellent composition for inhibiting resistance to an anticancer agent including a THBS1 inhibitor as an active ingredient.

The THBS1 inhibitor (suppressor) of the present invention may include any agent or means known in the art, as long as the THBS1 inhibitor may reduce the object of the present invention, that is, the expression level or activity of THBS1 in cancer cells. For example, the inhibitor may include all inhibitors that inhibit the expression of a target gene THBS1 or the activity of a THBS1 protein, but is not limited thereto.

According to a preferred exemplary embodiment of the present invention, the THBS1 inhibitor may be at least one selected from the group consisting of antisense oligonucleotide, small interference RNA (siRNA), short hairpin RNA (shRNA), microRNA (miRNA), and ribozyme that bind complementarily to mRNA of the THBS1 gene; or at least one selected from the group consisting of a compound, a peptide, a peptide mimetic, a substrate analog, an aptamer, and an antibody that specifically binds to the THBS1 protein, but is not limited thereto.

As used herein, the term “antisense nucleic acid” refers to DNA, RNA, or derivatives thereof containing a nucleic acid sequence complementary to a specific mRNA sequence, and serves to inhibit the translation to a protein of mRNA by binding to the complementary sequence in mRNA. The antisense sequence refers to a DNA or RNA sequence complementary to the mRNA of the gene and capable of binding to the mRNA, and may inhibit translation of the mRNA, translocation into the cytoplasm, maturation, or any other essential activity for overall biological functions.

In addition, the antisense nucleic acid may be modified at one or more base, sugar or backbone positions to enhance the efficacy. The nucleic acid backbone may be modified with phosphorothioate, phosphotriester, methyl phosphonate, short-chain alkyl, cycloalkyl, short-chain heteroatomic, heterocyclic intersugar linkages, and the like. In addition, the antisense nucleic acid may include one or more substituted sugar moieties. The antisense nucleic acid may include modified bases. The modified bases include hypoxanthine, 6-methyladenine, 5-methylpyrimidine (particularly, 5-methylcytosine), 5-hydroxymethylcytosine (HMC), glycosyl HMC, gentobiosyl HMC, 2-aminoadenine, 2-thio uracil, 2-thiothymine, 5-bromouracil, 5-hydroxymethyluracil, 8-azaguanine, 7-deazaguanine, N6(6-aminohexyl) adenine, 2,6-diaminopurine, and the like. In addition, the antisense nucleic acid may chemically bind to one or more moieties or conjugates that improve the activity and cell adhesion of the antisense nucleic acid. The antisense nucleic acid includes fat-soluble moieties, such as cholesterol moiety, cholesteryl moiety, cholic acid, thioether, thiocholesterol, fatty chain, phospholipid, polyamine, polyethylene glycol chain, adamantane acetic acid, palmityl moiety, octadecylamine, hexylaminocarbonyl-oxycol esterol moiety, and the like, but is not limited thereto. The antisense oligonucleotide may be synthesized in vitro by a conventional method to be administered in vivo, or may be synthesized in vivo.

As used herein, “siRNA” means a nucleic acid molecule capable of mediating RNA interference or gene silencing. Since the siRNA may inhibit the expression of a target gene, the siRNA is provided as an efficient gene knock-down method or a gene therapy method.

The siRNA molecule of the present invention may have a structure forming a double chain in which a sense strand (a sequence corresponding to an mRNA sequence of a THBS1 gene as a target gene) and an antisense strand (a complementary sequence to the mRNA sequence) are located opposite each other, and the siRNA molecule of the present invention may have a single-stranded structure with self-complementary sense and antisense strands. Furthermore, siRNA may include a part which is not paired by mismatch (corresponding bases are not complementary), bulge (there is no corresponding base on one chain), etc. without limiting that a double-stranded RNA part pairing RNAs is completely paired. In addition, when a siRNA end structure may suppress the expression of the target gene by an RNAi effect, both a blunt end and a cohesive end are possible, and the cohesive end structure can be both a 3′-end protruding structure and a 5′-end protruding structure.

The “shRNA” of the present invention is called small hairpin RNA or short hairpin RNA, and is used for silencing the gene by RNA interference. Usually, the shRNA is introduced into a target cell using a vector. Such a shRNA hairpin structure is also cleaved by other intracellular substances to become siRNA.

In an exemplary embodiment of the present invention, short hairpin RNA (shRNA) represented by SEQ ID NO: 1 was used as the THBS1 inhibitor, but it is not limited thereto as long as the purpose of the present invention can be achieved.

In addition, the present invention may include a functional equivalent of the shRNA base sequence represented by SEQ ID NO: 1. The “functional equivalent” refers to polynucleotide that exhibits substantially the same physiological activity as the polynucleotide represented by the base sequence set forth in SEQ ID NO: 1 by having a sequence homology of at least 70% or more, preferably 80% or more, more preferably 90% or more, much more preferably 95% or more, as a result of deletion, substitution or insertion of bases. The “% of sequence homology” with the polynucleotide is determined by comparing two optimally arranged sequences with a comparison region, and a part of a polynucleotide sequence in the comparison region may include addition or deletion (i.e., gap) compared to a reference sequence (without including addition or deletion) for an optimal alignment of the two sequences.

In other words, the present invention is significant in the sense that it is found that when a THBS1 inhibitor and an anticancer agent are administered in combination, it is possible to overcome resistance or drug resistance induced to the anticancer agent and to significantly improve the anticancer effect of conventional anticancer agents. Accordingly, it will be apparent to those skilled in the art that the THBS1 inhibitor can be applied to the present invention regardless of its type as long as the inhibitor is used in the technical field of the present invention or has been found to have THBS1 inhibitory activity, and is not limited to specific types.

In addition, in the present invention, the THBS1 inhibitor may be administered simultaneously or sequentially with an anticancer agent.

In the present invention, the anticancer agent can be used without restrictions as long as the anticancer agent is effective in treating cancer, but preferably may be characterized as a p53 activator, which is a targeted anticancer agent. The p53 activator may be at least one selected from the group consisting of APR-246 (Eprenetapopt, PRIMA-1MET), CP-31398, PK083, PK11007, NSC319726 (ZMC1), stictic acid, Nutlin-3a, RO6839921, NSC 146109 hydrochloride, RITA, Tenovin-1, HLI373, WR 0165, Idasanutlin, and YH 239-EE, but is not limited thereto.

In an exemplary embodiment of the present invention, APR-246 (Eprenetapopt, PRIMA-1MET) was used as the p53 activator, but it is not limited thereto.

In addition, there is provided a composition for enhancing responsiveness to an anticancer agent including a THBS1 inhibitor as an active ingredient.

In other words, when the composition is treated in combination with cells having resistance inhibition and sensitivity, the composition may enhance the responsiveness to the anticancer agent to lower the dose of the anticancer agent used, and improve side effects of the anticancer agent.

In addition, according to another aspect of the present invention, the present invention provides an anticancer adjuvant including the composition according to the present invention.

As used herein, the term “anticancer adjuvant” refers to an agent that can alleviate, improve, or increase the anticancer effect of the anticancer agent by administering it in combination with the anticancer agent when administering the anticancer agent.

In the present invention, the anticancer adjuvant may be used as an anticancer agent or anticancer adjuvant depending on a treatment concentration, and may enhance the sensitivity (susceptibility) of the anticancer agent.

The adjuvant of the present invention may be administered simultaneously, separately, or sequentially with or from the anticancer drug. The order of administration of the anticancer adjuvant according to the present invention, that is, which of the anticancer agent and the anticancer adjuvant is administered at some point in time and simultaneously, individually, or sequentially, may be determined by a doctor or expert. The order of administration may vary depending on many factors. The anticancer adjuvant may be administered in combination with a known compound that has the effect of preventing, alleviating, or treating cancer. In this regard, the anticancer adjuvant may be administered simultaneously or sequentially with known compounds.

In an exemplary embodiment of the present invention, it was confirmed that the THBS1 inhibitor may increase the responsiveness of the p53 activator in a cancer cell line with a p53 mutation, so that it was confirmed that the THBS1 inhibitor according to the present invention can be used as an anticancer adjuvant.

In addition, according to yet another aspect of the present invention, the present invention provides a pharmaceutical composition for preventing or treating cancer including a THBS1 inhibitor and an anticancer agent as active ingredients.

In the present invention, it was confirmed that when the inhibitor for THBS1 is used together with a p53 activator in a cancer cell line with a p53 mutation, an increased therapeutic effect is achieved by activating the action of p53 known as a tumor suppressor protein. Accordingly, a combination including the THBS1 inhibitor and the anticancer agent of the present invention is provided as an effective combination administration strategy therapy in various cancer types.

Therefore, the present invention provides a method for inhibiting resistance to an anticancer agent, including administering to a subject a composition for inhibiting resistance to an anticancer agent including a Thrombospondin 1 (THBS1) inhibitor as an active ingredient.

In addition, the present invention provides a method for treating cancer including administering to a subject a pharmaceutical composition for preventing or treating cancer including a THBS1 inhibitor and an anticancer agent as active ingredients.

The effect of preventing and/or treating cancer includes not only the effect of inhibiting the growth of cancer cells, but also the effect of inhibiting the worsening of cancer due to migration, invasion, metastasis, etc.

In the present invention, the cancer may include any cancer as long as the composition of the present invention achieves the desired effect. For example, the cancer may be at least one selected from the group consisting of lung cancer, liver cancer, colon cancer, adrenal cancer, stomach cancer, breast cancer, blood cancer, bone cancer, pancreatic cancer, skin cancer, head or neck cancer, cutaneous or intraocular melanoma, uterine sarcoma, ovarian cancer, rectal cancer, anal cancer, fallopian tube carcinoma, endometrial carcinoma, cervical cancer, small intestine cancer, endocrine cancer, thyroid cancer, parathyroid cancer, soft tissue tumor, urethral cancer, prostate cancer, bronchogenic cancer, and bone marrow tumor. In an exemplary embodiment of the present invention, the cancer was lung cancer, but is not limited thereto.

The cancer may be p53 mutation cancer.

As used herein, the term “including as the active ingredient” means including the THBS1 inhibitor, which is the active ingredient of the present invention, in an amount sufficient to achieve a predetermined efficacy or activity. The THBS1 inhibitor may be administered in a pharmaceutically effective dose, and the effective dose level may be determined depending on the type, age, and sex of a subject, sensitivity to a drug, a treatment period, drugs used simultaneously, and other medical factors.

In the present invention, the pharmaceutical composition may be in the form of capsules, tablets, granules, injections, ointments, powders or beverages, and the pharmaceutical composition may target humans. The pharmaceutical composition is not limited thereto, but may be formulated and used in the form of oral formulations, such as powders, granules, capsules, tablets, aqueous suspensions, etc., external preparations, suppositories, and sterile injectable solutions according to a general method, respectively.

The pharmaceutical composition according to the present invention may include a pharmaceutically acceptable carrier. The pharmaceutically acceptable carrier may be used with a binder, a slip modifier, a disintegrant, an excipient, a solubilizer, a dispersant, a stabilizer, a suspending agent, a pigment, a flavoring, and the like during oral administration, may be mixed and used with a buffering agent, a preservative, a painless agent, a solubilizer, an isotonic agent, a stabilizer, and the like in the case of injections, and may be used with a base, an excipient, a lubricant, and a preservative, and the like in the case of topical administration. The formulations of the pharmaceutical composition according to the present invention may be prepared variously in combination with the pharmaceutically acceptable carrier described above. For example, for oral administration, the pharmaceutical composition may be formulated in the form of tablets, troches, capsules, elixirs, suspensions, syrups, and wafers, and for injections, the pharmaceutical composition may be formulated into a single dose ampoule or a multiple dose form.

Meanwhile, examples of the carrier, the excipient, and the diluent suitable for the formulations may be used with lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oils, or the like. In addition, the pharmaceutical composition may further include fillers, anti-coagulating agents, lubricants, wetting agents, flavorings, emulsifiers, preservatives, and the like.

The route of administration of the pharmaceutical composition according to the present invention is not limited thereto, but includes oral, intravenous, intramuscular, intraarterial, intramedullary, intrathecal, intracardiac, transdermal, subcutaneous, intraperitoneal, intranasal, intestinal, topical, sublingual or rectal administration. The “parenteral” includes subcutaneous, intradermal, intravenous, intramuscular, intraarticular, intrasynovial, intrasternal, intrathecal, intralesional and intracranial injection or infusion techniques. The pharmaceutical composition according to the present invention may also be administered in the form of a suppository for rectal administration.

The pharmaceutical composition according to the present invention may be variously changed according to various factors, including the activity of a specific active ingredient used, age, body weight, general health, sex, diet, administration time, administration route, excretion rate, drug formulation, and the severity of a specific disease to be prevented or treated. The dose of the pharmaceutical composition varies depending on the condition and body weight of a patient, the degree of a disease, a drug form, and administration route and period, but may be appropriately selected by those skilled in the art. Preferably, the dose may be administered in an amount capable of obtaining the maximum effect with a minimum amount without side effects in consideration of all the factors, and an effective dose of more preferably 1 to 10000 μg/weight kg/day, much more preferably 10 to 1000 mg/weight kg/day may be repeatedly administered several times a day. The dose does not limit the scope of the present invention in any aspect.

The pharmaceutical composition of the present invention may be used alone or in combination with surgery, radiation therapy, hormone therapy, chemotherapy, and methods of using biological response modifiers, for prevention or treatment of target indications.

In the present invention, the p53 activator and the THBS1 inhibitor are administered in combination to exhibit an excellent anticancer effect capable of not only obtaining an excellent synergistic effect compared to using a single drug, but also obtaining an equivalent or greater effect compared to using a single drug even when the administration concentration is reduced and/or the administration interval is increased.

In other words, the p53 activator has a limitation in that high concentration treatment is required to exhibit anticancer efficacy. Such a limitation can be overcome by significantly lowering the minimum concentration at which an anticancer effect can be achieved by co-administration with the THBS1 inhibitor, as proposed in the present invention.

According to yet another aspect of the present invention, the present invention provides a food composition for inhibiting resistance to an anticancer agent including a THBS1 inhibitor as an active ingredient.

The food composition according to the present invention includes all forms, such as functional food, nutritional supplements, health food, health supplements, and food additives. The type of food composition may be formulated in any one form selected from the group consisting of powders, tablets, capsules, pills, and liquids according to a conventional method known in the art, but is not limited thereto. The food composition may be formulated in various forms using methods known in the art.

For example, as the health food, the THBS1 inhibitor itself of the present invention may be taken in by granulation, encapsulation and powder or prepared in the form of tea, juice, and drinks to be drunk. In addition, the THBS1 inhibitor of the present invention may be mixed with a known substance or active ingredient known to have prevention, alleviation, or treatment activity of cancer to be prepared in the form of a composition.

In addition, the functional food may be prepared by adding the THBS1 inhibitor of the present invention to beverages (including alcoholic beverages), fruits and processed foods thereof (e.g., canned fruit, bottled food, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, malt candies, dairy products (e.g., butter, cheese, etc.), edible vegetable oil, margarine, vegetable protein, retort food, frozen food, various seasonings (e.g., soybean paste, soy sauce, sauce, etc.), etc.

In addition, the food composition of the present invention may include conventional food additives, and the suitability as the “food additive” is determined by the specifications and standards for the corresponding item in accordance with the general rules of the Food Additive Codex, general test methods, and the like approved by the Food and Drug Administration, unless otherwise specified. The items disclosed in the “Korean Food Additives Codex” may include, for example, chemical composites such as ketones, glycine, potassium citrate, nicotinic acid, cinnamic acid, etc.; natural additives such as desensitizing dye, licorice extract, crystal cellulose, Kaoliang color, guar gum, etc.; mixed formulations such as sodium L-glutamic acid formulations, alkali agents for noodles, preservative formulations, tar color formulations, etc.

In the food composition of the present invention, the THBS1 inhibitor may preferably be included in an amount of 0.00001 to 50 wt % based on the food composition. If the content is less than 0.00001 wt %, the effect is insignificant, and if the content exceeds 50 wt %, an increase in effect compared to the amount used is minimal, which is uneconomical.

In addition, in order to use the THBS1 inhibitor of the present invention in the form of a food additive, the THBS1 inhibitor may be prepared and used in the form of tablets, capsules, powders, granules, liquids, pills, etc.

When the composition of the present invention is prepared as beverages, like general beverages, the composition may include various flavoring agents or natural carbohydrates as an additional ingredient. As the above-mentioned natural carbohydrates, monosaccharides such as glucose and fructose, disaccharides such as maltose and sucrose, natural sweeteners such as dextrin and cyclodextrin, synthetic sweeteners such as saccharin and aspartame, and the like may be used. A ratio of the natural carbohydrates may be generally about 0.01 to 10 g, preferably about 0.01 to 0.1 g per 100 ml of the composition of the present invention.

In addition to the ingredients, the composition of the present invention may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohol, a carbonating agent used in a carbonated drink, or the like. In addition, the composition of the present invention may include pulps for preparing natural fruit juices, fruit juice beverages and vegetable beverages. These ingredients may be used independently or in combination. Although the ratio of these additives is not greatly important, generally, the ratio thereof is selected in a range of 0.01 to 0.1 parts by weight per 100 parts by weight of the composition of the present invention.

As used herein, the “health supplement” or “health functional food” refers to food prepared and processed using raw substances or ingredients with functionality, which are useful for the human body according to the Health Functional Foods Act, and the “functionality” means intake for adjusting nutrients for the structures and functions of the human body or obtaining a useful effect on health applications such as physiological actions.

According to yet another aspect of the present invention, there is provided a screening method of substances for inhibiting resistance to an anticancer agent including the following steps:
(a) contacting a candidate substance with isolated cancer cells expressing THBS1;
(b) measuring the expression level of THBS1 in the cancer cells of step (a); and
(c) selecting the candidate substance as a substance for inhibiting resistance to an anticancer agent, when the expression level of THBS1 measured in step (b) is low compared to an untreated control group.

In the present invention, the method of measuring the expression level of THBS1, that is, the expression level of the THBS1 gene or the amount of THBS1 protein, may be performed by using a known technique, including a known process of isolating mRNA or protein from a biological sample. The biological sample refers to a sample collected from a living body of which the gene expression level or protein level is different from that of the control group. Examples of the sample may include tissue, cells, blood, serum, plasma, saliva, and urine, but are not limited thereto.

The expression level of the gene is preferably measured by measuring the level of mRNA, and methods for measuring the mRNA level include reverse transcription polymerase chain reaction (RT-PCR), real-time reverse transcription polymerase chain reaction, RNase protection assay, Northern blot, DNA chips, etc., but are not limited thereto.

The protein level may be measured using an antibody. In this case, the marker protein in the biological sample and a specific antibody thereto form a combination, that is, an antigen-antibody complex, and the amount of the antigen-antibody complex formed may be measured quantitatively through the signal size of a detection label. The detection label may be selected from the group consisting of enzymes, fluorescent substances, ligands, luminescent substances, microparticles, redox molecules, and radioisotopes, but is not limited thereto. Analytical methods for measuring the protein levels include Western blot, ELISA, radioimmunoassay, radioimmunodiffusion, Ouchterony immunodiffusion, rocket immunoelectrophoresis, tissue immunostaining, immunoprecipitation assay, complement fixation assay, FACS, protein chips, etc., but are not limited thereto.

In the present invention, the candidate substance may be an individual nucleic acid or protein that is estimated or randomly selected to have the potential as a medicine that inhibits the activity or expression level of the THBS1 protein or the gene encoding the THBS1 protein according to a conventional selection method, other extracts or natural products, compounds, etc.

In an exemplary embodiment of the present invention, it was confirmed that when the THBS1 inhibitor is administered in combination with the p53 activator, it is possible to overcome resistance to the anticancer agent and increase the anticancer effect through a synergistic effect. Substances that reduce the activity or expression level of the THBS1 protein or gene can be used as agents for inhibiting or improving resistance to the anticancer agent.

According to yet another aspect of the present invention, there is provided an information providing method for determining resistance to a p53 activator, including the following steps:
(a) measuring the expression level of THBS1 and mutation of p53 in a sample isolated from a subject; and
(b) determining that the subject has resistance to a p53 activator when the expression level of THBS1 is higher than that of a control sample and a mutation occurs in p53.

In this specification, “the information providing method for determining resistance to the p53 activator” refers to a method of providing information on a therapeutic effect of the p53 activator when the p53 activator is used as an anticancer agent for cancer treatment, and a method of providing information on whether resistance to the p53 activator is not exhibited, whether cancer progression may be effectively inhibited, whether cancer cells may be killed, or the like.

Preferably, the information providing method means a method of providing information that a p53 activator cannot be used for effective cancer treatment when cancer patients have a mutation in a p53 gene, the p53 function does not function normally, and THBS1 is expressed at a high level compared to a control sample. Since the method of the present invention uses the THBS1 described above, the description of duplicated contents will be omitted to avoid excessive complexity of the present specification.

### Advantageous Effects

According to the present invention, a THBS1 inhibitor inhibits the drug resistance of a target anticancer agent and thus increases an anticancer effect when administered in combination with the target anticancer agent. Accordingly, the present invention can overcome resistance to a targeted anticancer agent and increase a treatment success rate of anticancer agents for cancer patients, thereby suggesting new possibilities for treatment strategies using target anticancer agents and contributing to the realization of precision medicine.

### DESCRIPTION OF DRAWINGS

FIG. 1 schematically shows a process of discovering a combination target to overcome resistance to a targeted anticancer agent of the present invention. (1) shows pathway information obtained from a database, gene expression levels of cell lines, and drug response information. (2) shows sensitive cell lines and resistant cell lines divided based on the drug response information of the cell lines obtained in (1). (3) shows a process of learning an anomalous gene detection using generative adversarial networks and graph neural networks for overcoming drug resistance (AnoDAN) deep learning model presented in the present invention using the sensitive cell line data in (2). (4) shows a process of calculating anomaly scores at pathway, sample, and gene levels by inputting resistant cell lines of (2) into the model learned in (3). (5) selects a pathway causing resistance using anomaly scores in (4), and shows anomaly scores for each gene in the pathway. (6) shows a combination target gene to overcome resistance selected based on a high gene anomaly score in the pathway selected in (5). (7) shows a process of verifying a change in drug responsiveness and a resistance occurrence mechanism through controlling the expression level of the target gene selected in (6).

FIG. 2 schematically shows a process of identifying effective resistance-overcoming combination target and mechanism of the present invention. FIG. 2A shows AnoDAN with a new deep neural network (DNN) structure and a learning process using generative adversarial networks (GAN) and graph neural networks (GNN) using gene expression and pathway data together. FIG. 2B shows a method of assigning anomaly scores at a gene level, a pathway level, and a sample level using differences between actual data and generated data. FIG. 2C shows that a model has been learned by converging loss functions of each of a generator, a discriminator, and an encoder that configure the proposed AnoDAN. FIG. 2D shows that the model has been learned through well-overlapping when actual data and data generated from the model in FIG. 2C were mapped in two dimensions using uniform manifold approximation and projection (UMAP).

FIG. 3 shows results of outputting sample-level anomaly scores calculated for drug-sensitive cell lines and drug-resistant cell lines as a learning model for discovering combination targets to overcome resistance to a targeted anticancer agent of the present invention.

FIG. 4 shows results of performing learning using known trametinib data to confirm the reliability of the learning model of the present invention. FIG. 4A shows that a model has been learned by converging loss functions of a generator, a discriminator, and an encoder. FIG. 4B shows that the model has been learned through well-overlapping when actual data and data generated from the model in FIG. 4A were mapped in two dimensions using UMAP. FIG. 4C shows the ranking of pathways with high anomaly scores for resistance to trametinib.

FIG. 5 shows a process of selecting THBS1, a gene with a high anomaly score and commonly belonging to a p53 signaling pathway and a TGF-beta signaling pathway obtained by being applied to a learning model of the present invention as a combination target to overcome target drug resistance. FIG. 5A shows pathway-level anomaly scores obtained by applying drug-resistant cell line data to a model previously learned as a sensitive cell line. FIG. 5B shows anomaly scores of each gene in a p53 signaling pathway, which has the highest anomaly score in FIG. 5A. At this time, the anomaly score is expressed as the degree of color darkness. FIG. 5C shows anomaly scores of genes in a TGF-beta signaling pathway, which has the second highest anomaly score in FIG. 5A. FIG. 5D shows that the THBS1 gene, which commonly belongs to the two signaling pathways in FIGS. 5B and 5C and has a high anomaly score, has been selected as a combination target to overcome target drug resistance.

FIG. 6 shows results of confirming the expression level of THBS1, selected as a combination target gene of the present invention, in cancer cell lines. FIG. 6A shows that on used data, the expression level of THBS1 is higher in drug-resistant cell lines than in drug-sensitive cell lines. FIG. 6B shows that when comparing THBS1 mRNA expression levels in resistant and sensitive cell lines in a directly performed experiment, the THBS1 mRNA expression level is higher in the resistant cell lines.

FIG. 7 shows results of confirming a drug response to a p53 activator in cancer cell lines with p53 mutations. FIG. 7A shows that in an actual experiment, an APR-246 drug-resistant cell line as a p53 activator hardly responds to the drug. FIG. 7B shows that when sensitive cell lines are treated with a drug, clear responses to the drug appear. FIG. 7C shows results of confirming the results of FIGS. 7A and 7B once again for drug responses by staining only living cells through a crystal violet experiment.

FIG. 8 shows results of regulating THBS1 expression and combining a p53 activator in drug-resistant cancer cell lines and drug-sensitive cancer cell lines, and results of experimentally verifying a resistance overcoming mechanism. FIG. 8A shows that the expression level of THBS1 is reduced in NCI-H1792 and NCI-H1793, which are APR-246 drug-resistant cell lines, and then the mRNA level is confirmed, so that the expression level is well reduced. FIG. 8B shows that the expression level of THBS1 is increased in NCI-H2009 and HCC827, which are highly drug-responsive cell lines, and then the mRNA level is confirmed, so that the expression level is well increased. FIG. 8C shows that when THBS1 expression is inhibited in resistant cell lines, it is verified through a cell viability graph and a crystal violet experiment that drug responsiveness is further increased. FIG. 8D shows that when THBS1 expression is increased in drug-sensitive cell lines, the cells are drug-resistant. FIG. 8E shows a positive feedback relation between THBS1 and TGF-beta signaling pathways that activate each other. At this time, when comparing the protein expression levels of TGFβ1 and p-SMAD2/3 to confirm the positive feedback relation, it is found that the combined treatment of the drug and THBS1 in resistant cell lines decreases the protein expression in the TGF-beta signaling pathway. FIG. 8F shows results of experimentally verifying a positive feedback relation through the increase in protein expression in the TGF-beta signaling pathway during combined treatment when the same experiment is performed on drug-sensitive cell lines. FIG. 8G shows that the positive feedback observed in the previous experiment is subjected to processes in which THBS1 activates TGFβ1, and through TGFβR, p-SMAD2/3 is activated and then THBS1 is expressed again. FIG. 8H shows that drug resistance occurs due to positive feedback between THBS1 and TGF-beta signaling pathways when APR-246 is treated alone, so that the drug responsiveness may be increased by combined treatment to inhibit THBS1.

FIG. 9 shows results of confirming effects depending on a concentration when combined with a THBS1 inhibitor and a p53 activator in drug-sensitive cell lines of the present invention. FIGS. 9A and 9B show results of observing cell viability after inhibiting THBS1 in sensitive cell lines.

FIG. 10 shows results of confirming positive feedback between the THBS1 and TGF-beta signaling pathways of the present invention. FIG. 10A shows that the mRNA expression level of TGFβ1 is higher in drug-resistant cell lines than in sensitive cell lines. FIG. 10B shows that when THBS1 is inhibited in the resistant cell lines, TGFβ1 is also reduced. FIG. 10C shows that the activity score of the TGF-beta signaling pathway is higher in the resistant cell lines on Signaling Pathway Enrichment using Experimental Datasets (SPEED) data provided by a GDSC database. FIGS. 10D and 10E show that when cells are seeded in two sensitive cell lines and treated with a drug for activating the TGF-beta signaling pathway 24 hours later, the expression level of THBS1 is also increased while the mRNA expression level of TGFβ1 is increased.

### BEST MODE OF THE INVENTION

Hereinafter, Examples are to describe the present invention in more detail, and it will be apparent to those skilled in the art that the scope of the present invention is not limited by these Examples in accordance with the gist of the present invention.

### Experimental Methods and Materials

### Cell Culture

Human lung cancer cell lines NCI-H1793, NCI-H2009, and HCC827 were purchased from the Korea Cell Line Bank (KCLB), and NCI-H1792 was purchased from the American Type Culture Collection (ATCC). NCI-H1793 and HCC827 were cultured in a RPMI 1640 medium supplemented with 10% fetal bovine serum (FBS) and antibiotics (100 U/ml penicillin, 100 μg/ml of streptomycin, and 0.25 μg/ml of Fungizone). NCI-H1792 and NCI-H2009 were cultured with the same supplements in a DMEM medium. All the cell lines were cultured at 37° C. in an incubator containing 5% carbon dioxide.

### Reagents

Dimethyl sulfoxide (DMSO) was purchased from Sigma-Aldrich and was used as a drug control. APR-246 and TGFβ1 protein were purchased from MedChemExpress (MCE). APR-246 was used by diluting 25 mg to a final concentration of 10 mM, and the cells were treated for 4 to 5 days when checking the cell viability and for 24 hours when performing a Western blot experiment. In addition, TGFβ1 at a concentration of 10 μg/ml was a TGF-beta pathway activator, and drug-sensitive cell lines were seeded and treated with 5 ng/ml of TGFβ1 24 hours later, and the cells were harvested 24 hours later.

### Virus Production for Gene Knockdown and Overexpression Experiments

HEK 293T cells were used to transfect shRNA targeting THBS1 and an overexpression vector. In addition to shRNA (shTHBS1; GTAGGTTATGATGAGTTTAAT; SEQ ID NO: 1) and an overexpression vector (pDNA (VB220725-1808zjh); VectorBuilder), scramble and packaging mixes pLP1, pLP2, and pLP/VSVG were used together with lipofectamine to produce lentiviruses. 48 hours after transfection, target cell lines NCI-H1792, NCI-H1793, NCI-H2009, and HCC827 were transduced using a viral product and 4 μg/ml of polybrene. Infected cells were selected using 1 μg/ml of puromycin for at least one week.

### Cell Growth Analysis and Crystal Violet Analysis

Cells were seeded in a 96-well plate at 3 to 5×10³ cells per well. A drug was then treated within 24 hours after seeding. After drug treatment, cell growth was recorded every 3 hours using IncuCyte ZOOM for 4 to 5 days. Cell viability was analyzed using IncuCyte ZOOM 2016A software. After 4 to 5 days, the cells were stained with 1% crystal violet for 30 minutes at room temperature and washed with distilled water to confirm the amount of viable cells.

Total RNA Extraction and Real-Time Polymerase Chain Reaction (qRT-PCR)

Total RNA was extracted from cells using a total RNA extraction kit from Intron Biotechnology. Genomic DNA was removed by treatment with RNase-free DNase I. Complementary DNA (cDNA) was synthesized using a DiaStar RT kit of Solgent. In addition, RT-PCR was performed using Veriti 96-well Thermal Cycler of Applied Biosystems, and real-time polymerase chain reaction (qRT-PCR) was performed using a QuantStudio 5 real-time PCR system, an SYBR master mix (Genet Bio), and primers. The sequences of qRT-PCR primers used at this time were listed in Table 1 below.

TABLE 1

| | Forward | Reverse |
|---|---|---|
| Target | primer sequence | primer sequence |
| gene | (5′-3′) | (5'-3') |
| GAPDH | TGATGACATCAAGAA | TCCTTGGAGGCCATG |
| | GGTGGTGAAG | TGGGCCAT |
| | (SEQ ID NO: 2) | (SEQ ID NO: 3) |
| THBSI | QGGGCGTCAATGACA | TCACCACGTTGTTGT |
| | ATTTCCAG | CAAGGGT |
| | (SEQ ID NO: 4) | (SEQ ID NO: 5) |
| TGF | TACCTGAACCCGTGT | GTTGCTGAGGTATCG |
| β 1 | TGCTCTC | CCAGGAA |
| | (SEQ ID NO: 6) | (SEQ ID NO: 7) |

### Western Blot

Cells were harvested 24 hours after drug treatment, washed with phosphate-buffered saline (PBS), and lysed with a lysis buffer (20 mM HEPES (pH 7.2), 150 mM NaCl, 0.5% Triton X-100, 10% glycerol, 0.1% SDS) and 0.1% protease inhibitor and phosphatase inhibitor cocktail. A Western blot experiment was performed using anti-THBS1 (sc-59887), anti-TGFβ1 (sc-130348), anti-SMAD2/3 (sc-133098), mouse IgG (sc-2025), and rabbit IgG (sc-2027) antibodies from Santa Cruz Biotechnology Inc. and an anti-phospho-SMAD2/3 (#8828) antibody from Cell Signaling Technology Inc.

### Example 1. Discovery of Combination Targets to Overcome Resistance to Targeted Anticancer Agents

### 1-1. Construct of Discovery Model

To discover effective combination targets to overcome resistance to targeted anticancer agents, the present inventors presented a methodology for performing anomaly detection analysis by applying gene expression information and pathway information to a Generative Adversarial Network (GAN) model based on a signaling database. Typically, an experiment was conducted on APR-246, a p53-targeting agent that activated p53 as a targeted anticancer agent.

This was briefly described as follows: Data obtained from the Genomics of Drug Sensitivity in Cancer (GDSC) and Kyoto Encyclopedia of Genes and Genomes (KEGG) databases were used. The KEGG database was used for pathway information, but other databases that provide linkage information between genes can also be used. The anomaly detection analysis was conducted in the following three steps. First, a GAN model was learned to simulate gene expression information of APR-246 drug-sensitive cell lines. At this time, GNN was applied to a discriminator of the GAN model to distinguish at the pathway level whether the gene expression information was actual gene expression information or gene expression information generated by a generator. Second, an encoder was learned to embed the expression information of APR-246 drug-sensitive cell lines into a latent space of the pre-learned GAN. Third, the gene expression information of the APR-246 drug-resistant cell lines was regenerated using a pre-learned encoder and the generator of the GAN model, and then a difference from the actual expression level was calculated to detect anomaly values. By using pathway information in the learning process, the method can utilize richer biological information to find resistance-overcoming genes, and as a result, it is possible to identify the pathway-level resistance-overcoming mechanism. Using this method, it was found that the p53 signaling pathway and the TGF-beta signaling pathway with the top anomaly score were a cause of resistance to the APR-246 drug, and THBS1, which had a high anomaly score in both the signaling pathways, was discovered as a target gene to overcome drug resistance.

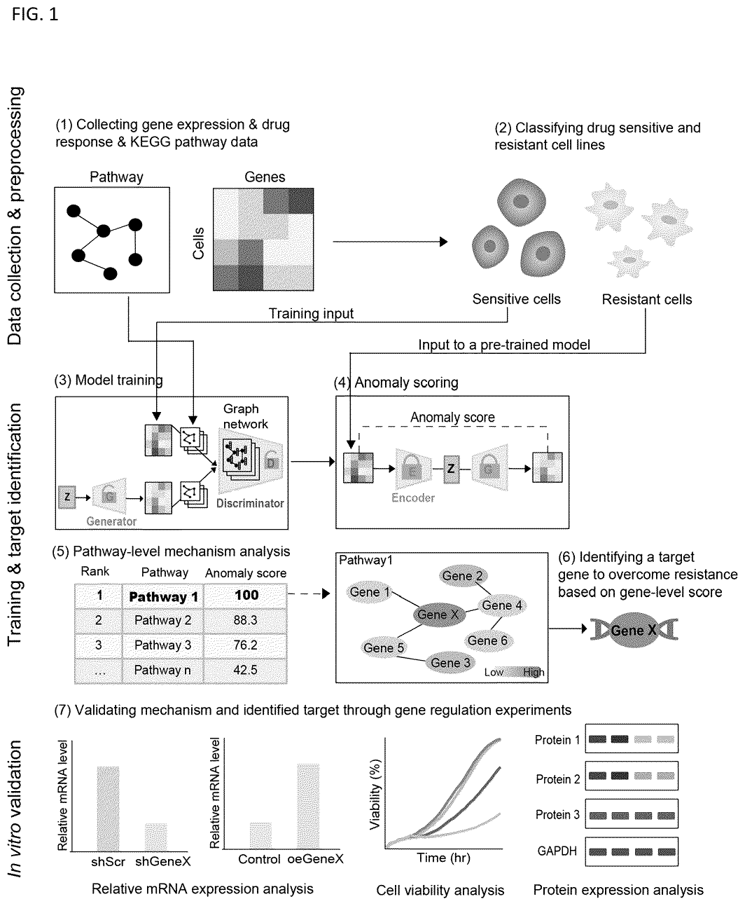

More specifically, data obtained from GDSC and KEGG databases were divided into sensitive cell lines and resistant cell lines based on IC₅₀ values for drugs. A model called anomalous gene detection using generative adversarial networks and graph neural networks for overcoming drug resistance (AnoDAN) was learned using sensitive cell line data, and resistant cell lines were input into the learned model to obtain anomaly scores at pathway, sample, and gene levels. The pathway causing resistance was selected based on the anomaly scores, and a gene with a high anomaly score within the pathway was selected as a combination target to overcome resistance. Changes in drug responsiveness were observed by increasing or inhibiting the gene expression level of the selected target, and a resistance occurrence mechanism was experimentally verified (FIG. 1).

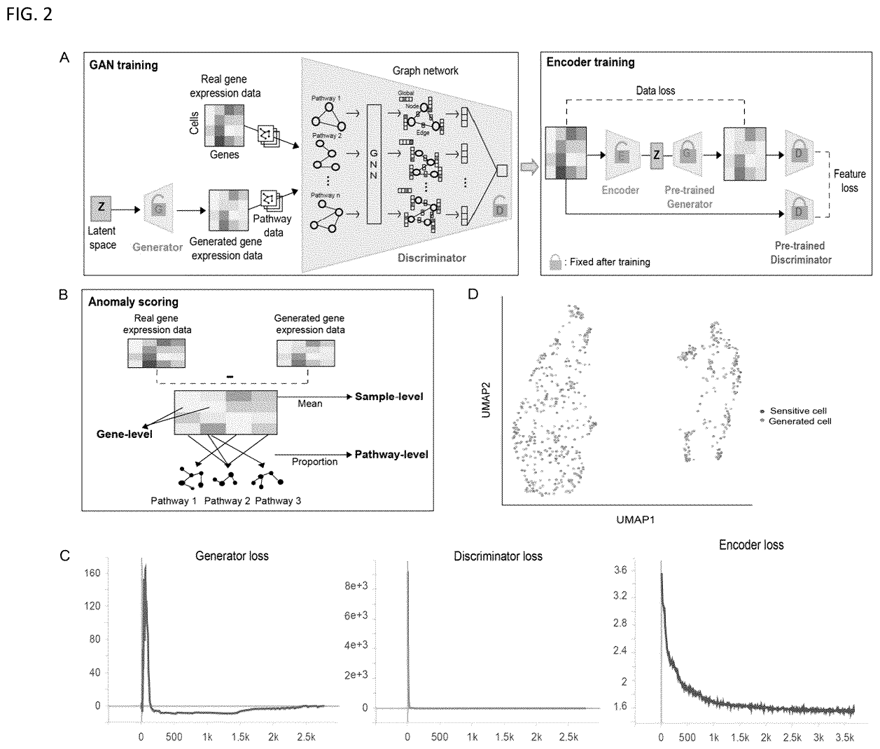

In addition, AnoDAN with a new DNN structure that utilized the advantages of GAN and GNN, was proposed to identify effective resistance-overcoming combination targets and mechanisms by using gene expression and pathway information together (FIG. 2A). In addition, to determine which pathway and gene caused occurrence of resistance, there was proposed a method of assigning anomaly scores using the difference between actual data and generated data (FIG. 2B). It was confirmed that learning was successful through the proper convergence of the loss function results of each of the generator, discriminator, and encoder (FIG. 2C). In addition, as a result of a dimensional reduction algorithm called Uniform Manifold Approximation and Projection (UMAP), it was confirmed that learning was performed well as the actual data and the generated data matched well with each other (FIG. 2D).

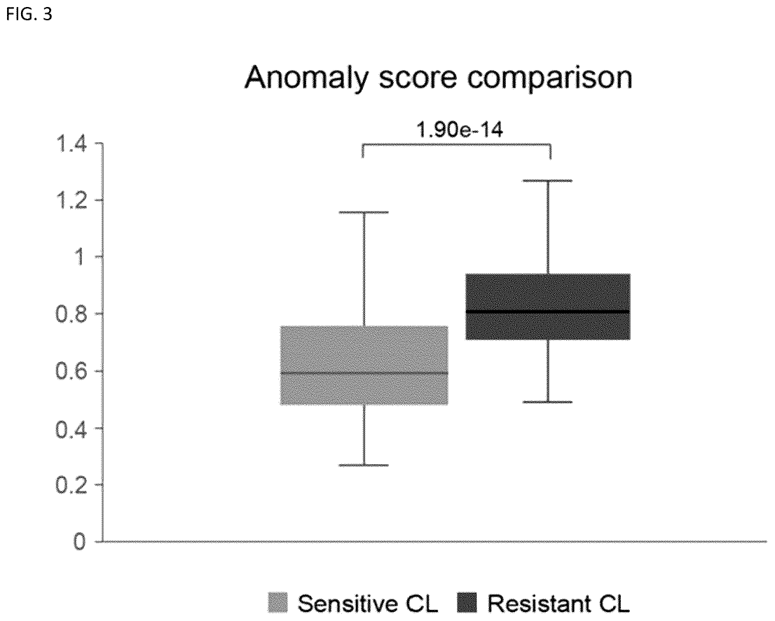

In addition, the anomaly scores presented above were calculated using the difference between the actual data and the generated data using pre-learned encoder and generator. Because the model was pre-learned using data from drug-sensitive cell lines, the generator had the ability to generate data that well simulated the characteristics of sensitive cell lines. Accordingly, when drug-resistant cell line data was input to the model, the generator did not generate data similar to the reality due to data from unseen distribution, so that the anomaly score was increased. As a result, as shown in FIG. 3, the anomaly scores of the drug-resistant cell lines were higher than the scores of the sensitive cell lines, and as a result, it was confirmed that the model was sufficiently learned to contain well the characteristics of the sensitive cell lines.

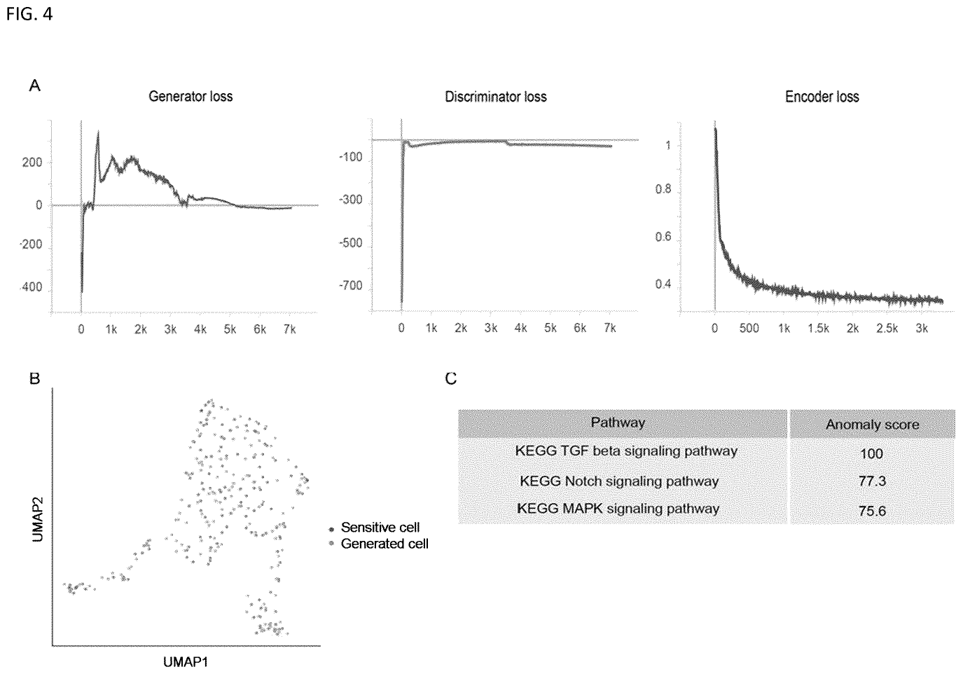

In addition, to confirm the reliability of the model, learning was performed through the same pipeline using data from trametinib, a well-known MEK inhibitor. It was confirmed that the model was learned well through the loss function results that converged properly (FIG. 4A). It was confirmed once again that model learning was sufficient through proper overlapping between the actual data and the generated data on UMAP (FIG. 4B). As it was well known as a drug highly related to the MAPK signaling pathway because of an MEK inhibitor, it was confirmed that the MAPK signaling pathway appeared as the upper pathway (FIG. 4C). Therefore, the reliability of the model and its applicability to other drugs were verified.

### 1-2. Discovery of Combination Drug Targets

The present inventors discovered a combination drug target that exhibited synergistic effects to ultimately achieve effective anticancer effects by targeting APR-246 among various p53 activators as a targeted anticancer agent to inhibit drug resistance to the APR-246 and enhance sensitivity, based on the discovery model constructed in Example 1-1 above.

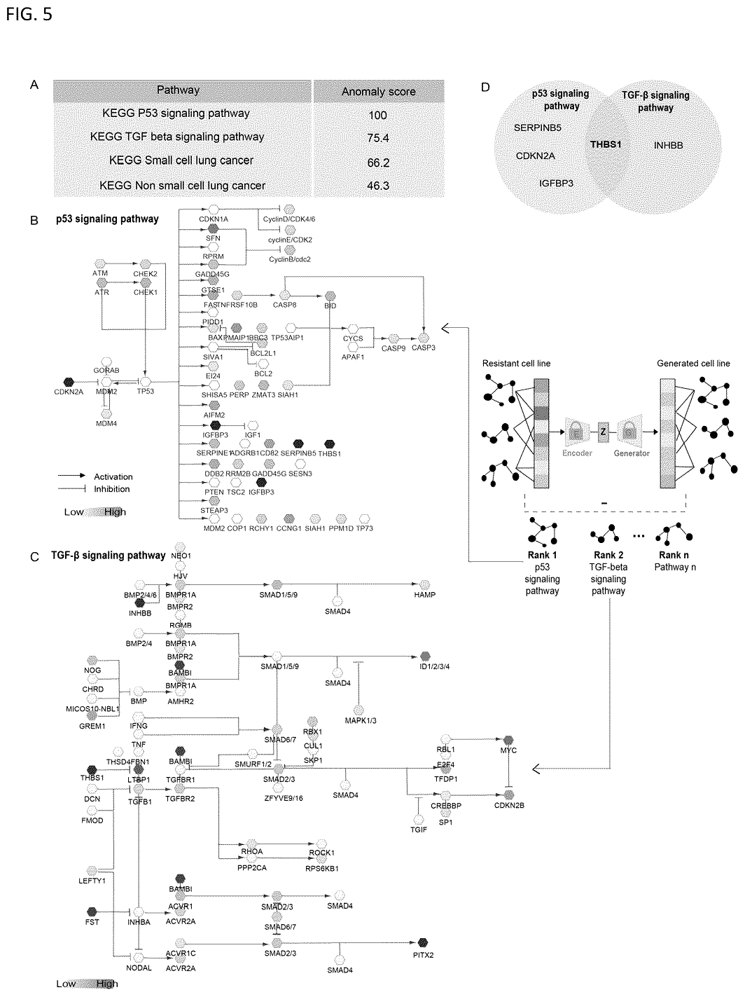

As a result, through the anomaly score result obtained by applying drug-resistant cell line data to a pre-learned model, it was confirmed that the p53 signaling pathway and the TGF-beta signaling pathway were the top pathways containing the most genes with high anomaly scores (FIG. 5A). The anomaly score of each gene in the P53 signaling pathway was expressed in terms of color darkness, and the top genes were selected based thereon (FIG. 5B). The top genes were selected through the same process even in the TGF-beta signaling pathway (FIG. 5C). THBS1, a gene that commonly belonged to the two signaling pathways and had a high anomaly score, was selected as a combination target to overcome APR-246 resistance (FIG. 5D).

### 1-3. Expression Levels in Cancer Cell Lines

The present inventors confirmed the expression level of THBS1, selected as the target gene in Example 1-2, in cancer cell lines.

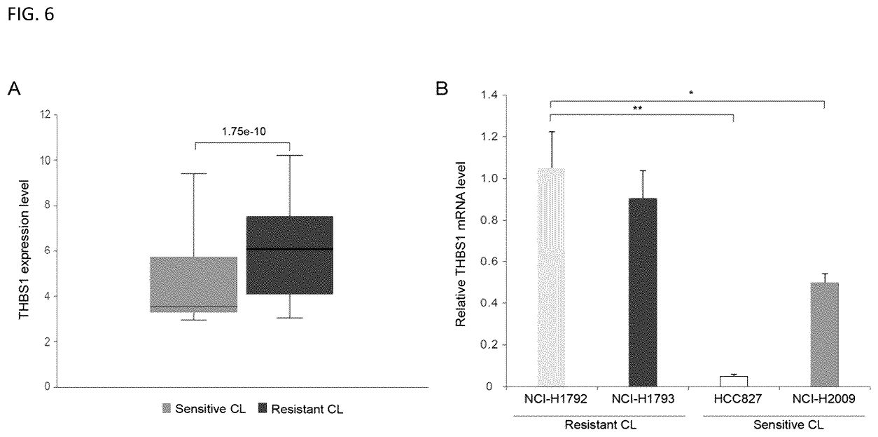

As a result, in the data used, the expression level of THBS1 was higher in APR-246 drug-resistant cancer cell lines than in sensitive cell lines (FIG. 6A). In addition, when comparing the THBS1 mRNA expression levels in lung cancer cell lines NCI-H1792 and NCI-H1793 resistant to the APR-246 drug and lung cancer cell lines HCC827 and NCI-H2009 sensitive to the APR-246 drug in actual experiments, the THBS1 mRNA expression level was observed to be higher in resistant cell lines (FIG. 6B). Therefore, it was confirmed that a strategy to inhibit THBS1 in resistant cell lines was required.

### 1-4. Drug Responses to p53 Activator in Cancer Cell Lines with p53 Mutations

The present inventors confirmed the cell viability according to the APR-246 drug treatment concentration for lung cancer cell lines NCI-H1793, NCI-H1792, HCC827, and NCI-H2009, which were known to have p53 mutations. At this time, APR-246 drug-sensitive cell lines and resistant cell lines were distinguished based on z-score normalized IC₅₀ values.

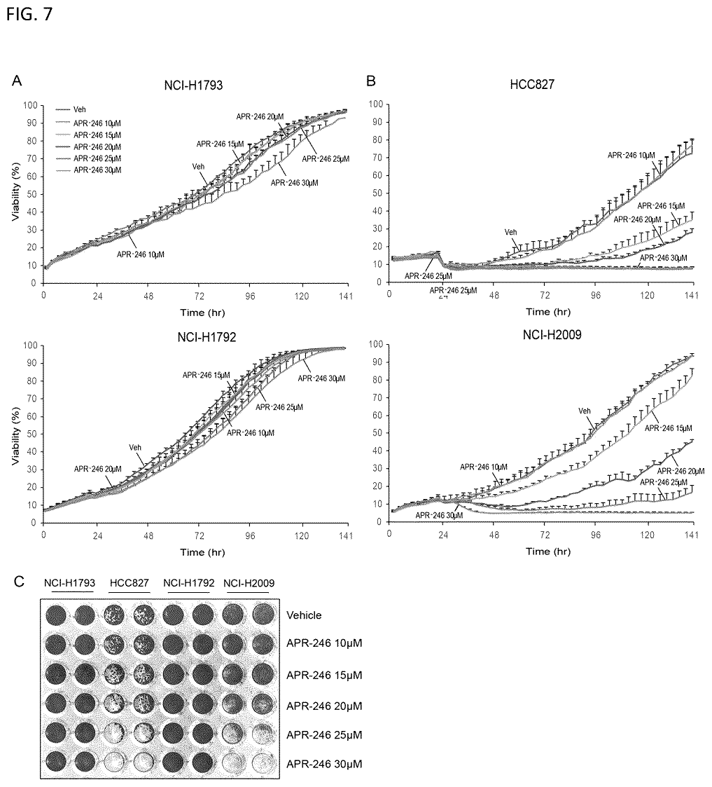

As a result, it was confirmed that NCI-H1793 and NCI-H1792 hardly responded even when treated with APR-246 (FIG. 7A). On the other hand, HCC827 and NCI-H2009 showed a clear response after treatment with APR-246 (FIG. 7B). The clear response was confirmed once again through crystal violet staining (FIG. 7C). Therefore, in the following Examples, NCI-H1793 and NCI-H1792 were used as the APR-246 drug-resistant cell lines, and HCC827 and NCI-H2009 cell lines were used as the APR-246 drug-sensitive (responsive) cell lines.

### Example 2. Confirmation of Effects and Resistance Mechanisms by Regulating THBS1 Expression and Combination of p53 Activator

To determine whether a combination of a THBS1 inhibitor and a p53 activator (targeted anticancer agent) may overcome drug resistance to the p53 activator, the present inventors measured cancer cell viability and crystal violet staining after treating lung cancer cell lines resistant to APR-246 and lung cancer cell lines sensitive to APR-246 with the THBS1 inhibitor and the p53 activator. In addition, THBS1 was overexpressed in NCI-H2009 and HCC827, lung cancer cell lines that were highly drug-responsive to APR-246, and the cell death effect due to overexpression was confirmed.

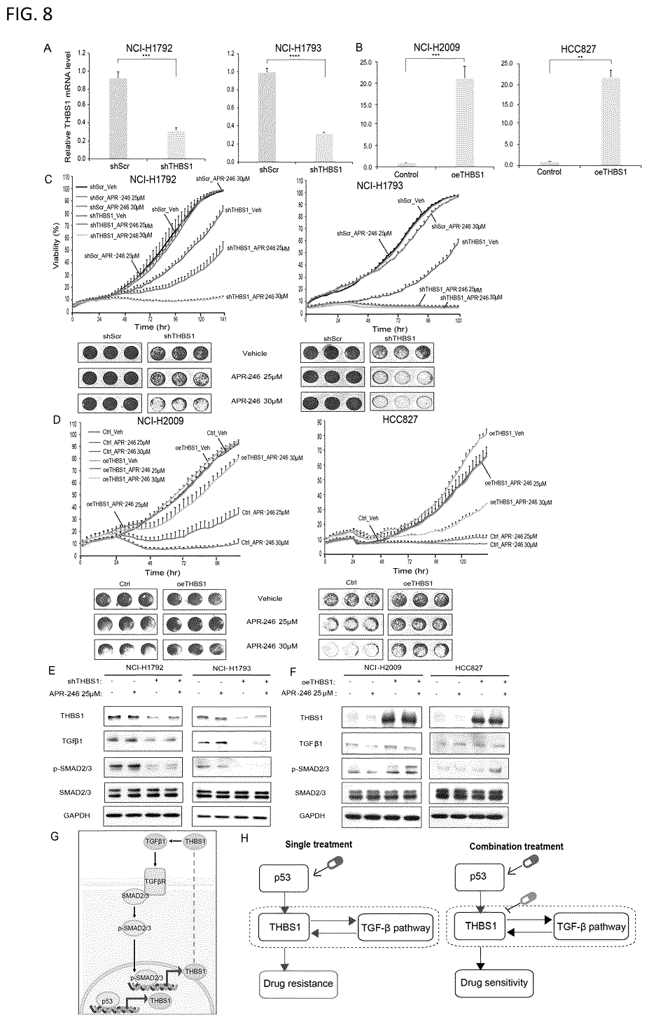

At this time, when NCI-H1792 and NCI-H1793 cell lines, lung cancer cell lines with drug resistance to APR-246, were treated with THBS1-targeting shRNA (SEQ ID NO: 1; GTAGGTTATGATGAGTTTAAT) as a THBS1 inhibitor, the THBS1 mRNA expression level was reduced (FIG. 8A). In addition, when THBS1 was overexpressed in NCI-H2009 and HCC827, lung cancer cell lines with high drug responsiveness to APR-246, it was confirmed that the THBS1 mRNA expression level was increased (FIG. 8B).

As a result, when APR-246 drug-resistant cancer cell lines were treated with the THBS1 inhibitor and the p53 activator APR-246, it was confirmed that while the expression of THBS1 was reduced by the THBS1 inhibitor, the drug responsiveness was further increased (FIG. 8C). On the other hand, when the expression of THBS1 was increased in APR-246-sensitive cancer cell lines, it was confirmed that the cancer cells exhibited drug resistance (FIG. 8D).

In addition, the present inventors compared the protein expression levels of TGFβ1 and p-SMAD2/3 to confirm the positive feedback relation between THBS1 and TGF-beta signaling pathways that activated each other.

As a result, it was confirmed that protein expression in the TGF-beta signaling pathway was decreased when the drug and THBS1 were treated in combination in resistant cell lines (FIG. 8E). In addition, the positive feedback relation was experimentally verified through an increase in protein expression in the TGF-beta signaling pathway when the drug-sensitive cell lines were treated with the drug and THBS1 in combination in the same manner (FIG. 8F). The positive feedback observed in the result was subjected to a series of processes in which THBS1 activated TGFβ1, and through TGFβR, p-SMAD2/3 was activated and then THBS1 was expressed again (FIG. 8G).

Therefore, when the APR-246 drug was treated alone, drug resistance occurred due to positive feedback between the THBS1 and TGF-beta signaling pathways, so that responsiveness to the drug may be increased by combined treatment with an inhibitor for inhibiting THBS1 (FIG. 8H).

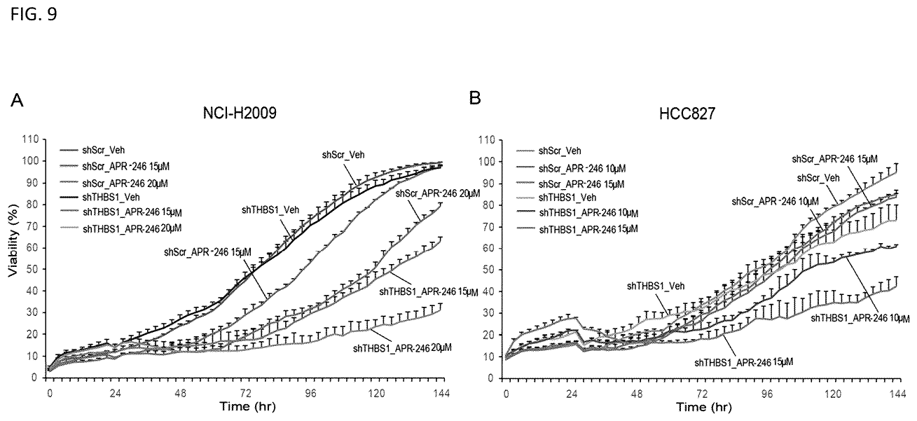

In addition, as a result of inhibiting THBS1 in sensitive cell lines, it was confirmed that drug responsiveness was further increased at low concentrations compared to a control group in which THBS1 was not inhibited (FIGS. 9A and 9B). These results suggest that even in cancer cells without resistance to p53, THBS1 inhibition may help in solving a drug toxicity problem that occurs when treating high concentrations of drugs.

Through the results, it was confirmed the combination of the p53 activator and the THBS1 inhibitor can overcome adaptive resistance to the drug (p53 activator) in cancer cells, and thus it is possible to provide an effective treatment method for cancer patients with abnormalities in the p53 signaling pathway.

### Example 3. Confirmation of Positive Feedback Between THBS1 and TGF-Beta Signaling Pathways

To further verify the positive feedback relation between the THBS1 and TGF-beta signaling pathways presented above, the present inventors measured the expression level of TGFβ1 according to THBS1 in APR-246 drug-resistant cell lines and sensitive cell lines.

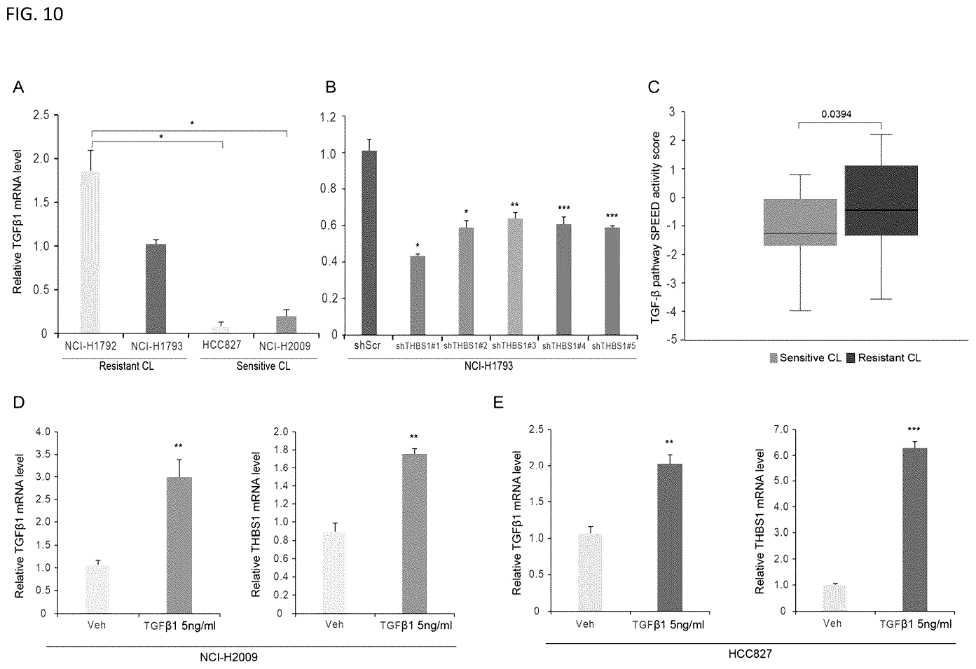

As a result, the mRNA expression level of TGFβ1 was higher in the APR-246 drug-resistant cell lines than in the sensitive cell lines (FIG. 10A). It was confirmed that when THBS1 was inhibited in the APR-246 drug-resistant cell lines, TGFβ1 was also reduced (FIG. 10B). In addition, it was confirmed that when the activity score of the TGF-beta signaling pathway was measured in the Signaling Pathway Enrichment using Experimental Datasets (SPEED) data provided by the GDSC database, the score was higher in the resistant cell lines (FIG. 10C). In other words, since the TGF-beta signaling pathway is more activated in resistant cell lines, it is proven that THBS1 may inhibit to lower its activity.

On the other hand, when cells were seeded in two types of sensitive cell lines and treated with a drug that activated the TGF-beta signaling pathway 24 hours later, it was confirmed that the expression level of THBS1 was also increased while the mRNA expression level of TGFβ1 was increased (FIGS. 10D and 10E).

Through the results, it was verified that the TGF-beta signaling pathway activity increased the activity of THBS1, and thus a positive feedback relation between the both was verified.

Overall, in the present invention, it was confirmed that the combined administration of a targeted anticancer agent (e.g., p53 activator) and an additional THBS1 inhibitor overcame the adaptive resistance of the targeted anticancer agent through a synergistic effect and improved its anticancer effect. In particular, the p53 activator and the THBS1 inhibitor showed an excellent synergistic effect in cancer cell lines with genetic mutations in the p53 signaling pathway, so that it was confirmed that therapy of combining the THBS1 inhibitor can be an effective strategy in various carcinomas.

Therefore, it is expected that the THBS1 inhibitor according to the present invention is used as a combination drug in an oncogenic signaling network with a feedback structure that causes drug resistance to overcome drug resistance of conventional targeted anticancer agents and increase the treatment success rate of targeted anticancer agents for cancer patients.

As described above, specific parts of the present invention have been described in detail, and it will be apparent to those skilled in the art that these specific techniques are merely preferred exemplary embodiments, and the scope of the present invention is not limited thereto. Therefore, the substantial scope of the present invention will be defined by the appended claims and their equivalents.

1. A method of inhibiting resistance to an anticancer agent, comprising administering to a subject in need thereof a composition for inhibiting resistance to an anticancer agent comprising a Thrombospondin 1 (THBS1) inhibitor.

2. The method for inhibiting resistance to the anticancer agent of claim 1, wherein the THBS1 inhibitor is selected from the group consisting of antisense oligonucleotide, small interference RNA (siRNA), short hairpin RNA (shRNA), microRNA (miRNA), and ribozyme that bind complementarily to mRNA of a THBS1 gene.

3. The method for inhibiting resistance to the anticancer agent of claim 1, wherein the THBS1 inhibitor is selected from the group consisting of a compound, a peptide, a peptide mimetic, a substrate analog, an aptamer, and an antibody that specifically bind to a THBS1 protein.

4. The method for inhibiting resistance to the anticancer agent of claim 1, wherein the THBS1 inhibitor is administered simultaneously or sequentially with an anticancer agent.

5. The method for inhibiting resistance to the anticancer agent of claim 1, wherein the anticancer agent is a p53 activator.

6. The method for inhibiting resistance to the anticancer agent of claim 5, wherein the p53 activator is selected from the group consisting of APR-246 (Eprenetapopt, PRIMA-1MET), CP-31398, PK083, PK11007, NSC319726 (ZMC1), stictic acid, Nutlin-3a, RO6839921, NSC 146109 hydrochloride, RITA, Tenovin-1, HLI373, WR 0165, Idasanutlin, and YH 239-EE.

7. The method for inhibiting resistance to the anticancer agent of claim 1, wherein be composition is for use in enhancing responsiveness to an anticancer agent.

8. The method resistance to the anticancer agent of claim 1, wherein the composition is for use in anticancer adjuvant.

9. A method for treating cancer, comprising administering to a subject in need thereof a pharmaceutical composition comprising a THBS1 inhibitor and an anticancer agent as active ingredients.

10. The method for treating cancer of claim 9, wherein the THBS1 inhibitor is at least one selected from the group consisting of antisense oligonucleotide, small interference RNA (siRNA), short hairpin RNA (shRNA), microRNA (miRNA), and ribozyme that bind complementarily to mRNA of a THBS1 gene.

11. The method for treating cancer of claim 9, wherein the THBS1 inhibitor is at least one selected from the group consisting of a compound, a peptide, a peptide mimetic, a substrate analog, an aptamer, and an antibody that specifically bind to a THBS1 protein.

12. The method for treating cancer of claim 9, wherein the anticancer agent is a p53 activator.

13. The method for treating cancer of claim 12, wherein the p53 activator is at least one selected from the group consisting of APR-246 (Eprenetapopt, PRIMA-1MET), CP-31398, PK083, PK11007, NSC319726 (ZMC1), stictic acid, Nutlin, RO6839921, NSC 146109 hydrochloride, RITA, Tenovin-1, HLI373, WR 0165, Idasanutlin, and YH 239-EE.

14. The method for treating cancer of claim 9, wherein the cancer is at least one selected from the group consisting of lung cancer, liver cancer, colon cancer, adrenal cancer, stomach cancer, breast cancer, blood cancer, bone cancer, pancreatic cancer, skin cancer, head or neck cancer, cutaneous or intraocular melanoma, uterine sarcoma, ovarian cancer, rectal cancer, anal cancer, fallopian tube carcinoma, endometrial carcinoma, cervical cancer, small intestine cancer, endocrine cancer, thyroid cancer, parathyroid cancer, soft tissue tumor, urethral cancer, prostate cancer, bronchogenic cancer, and bone marrow tumor.

15. The method for treating cancer of claim 14, wherein the cancer is p53 mutation cancer.

16. The method for treating cancer of claim 1, wherein the composition is for use in food composition for inhibiting resistance cancer agent.

17. A screening method of substances for inhibiting resistance to an anticancer agent comprising following steps:
(a) contacting a candidate substance with isolated cancer cells expressing THBS1;
(b) measuring an expression level of THBS1 in the cancer cells of step (a); and
(c) selecting the candidate substance as the substance for inhibiting resistance to the anticancer agent, when the expression level of THBS1 measured in step (b) is low compared to an untreated control group.

18. An information providing method for determining resistance to an anticancer agent, comprising following steps:
(a) measuring an expression level of THBS1 and mutation of p53 in a sample isolated from a subject; and
(b) determining that the subject has resistance to the anticancer agent when the expression level of THBS1 is higher than that of a control sample and a mutation occurs in p53.

19. (canceled)

20. (canceled)


SEQUENCE LISTING (7 sequences)

SEQ ID NO: 1 - RNA, 21 nt, synthetic construct
  source 1..21: mol_type = other RNA
gtaggttatgatgagtttaat

SEQ ID NO: 2 - DNA, 25 bp, synthetic construct
  source 1..25: mol_type = other DNA
tgatgacatcaagaaggtggtgaag

SEQ ID NO: 3 - DNA, 23 bp, synthetic construct
  source 1..23: mol_type = other DNA
tccttggaggccatgtgggccat

SEQ ID NO: 4 - DNA, 23 bp, synthetic construct
  source 1..23: mol_type = other DNA
ggggcgtcaatgacaatttccag

SEQ ID NO: 5 - DNA, 22 bp, synthetic construct
  source 1..22: mol_type = other DNA
tcaccacgttgttgtcaagggt

SEQ ID NO: 6 - DNA, 22 bp, synthetic construct
  source 1..22: mol_type = other DNA
tacctgaacccgtgttgctctc

SEQ ID NO: 7 - DNA, 22 bp, synthetic construct
  source 1..22: mol_type = other DNA
gttgctgaggtatcgccaggaa